(19) United States
(12) Patent Application Publication
(10) Pub. No.: US 2005/0113892 A1
(43) Pub. Date: May 26, 2005

(54) Surgical tool with an electroactive polymer for use in a body

(76) Inventor: Michael E. Sproul, Tequesta, FL (US)

Correspondence Address: MCHALE & SLAVIN, P.A., 2855 PGA BLVD, PALM BEACH GARDENS, FL, 33410, US

(21) Appl. No.: 10/724,369

(22) Filed: Nov. 26, 2003

(51) Int. Cl. A61F 2/00

(52) U.S. Cl. 607/100

(57) ABSTRACT

A surgical tool for doing work inside the body is powered by an electroactive polymer in the form of a transducer. The electroactive polymer is connected to an electrical power source and deforms from an initial position to a different second position upon electrical stimulation. The transducer can make a cavity in bone for internal splints or power a pump.

### BACKGROUND OF THE INVENTION

1. Field of the Invention

This invention is related to the field of surgery and, particularly, to the use of an electroactive polymer in a tool to accomplish work within the body. One example is in orthopedic surgery, as a bone tamp for bone fractures and in the procedure referred to as vertebroplasty. Another example is in variable volume implantable pumps to collect or deliver materials.

2. Description of the Prior Art

Vertebroplasty is a percutaneous technique for repairing spinal compression fractures by injecting bone cement into the vertebrae. The bone cement is used to shore up the collapsing vertebrae which relieves pain associated with undue pressure on the spinal nerves. The procedure is now broadened in application to osteoporotic patients as a surgical alternative to a regimen of narcotics and immobilization. A needle is inserted through the skin on a posterior-lateral tract and penetrates the hard shell of the vertebrae. A cannula is inserted over the needle and the needle is withdrawn leaving a pathway for the treatment material to be deposited within the marrow of the vertebral body. The material is inserted by either high pressure or low pressure mechanical, electrical or manual pumps. The procedure is monitored by fluoroscopy to monitor the injection to prevent the material from penetrating into the spinal canal or other unwanted areas.

Rather than using the injected material to form the cavity within the vertebrae, later devices use a balloon to form the space and control the spread of the bone cement. This gives in better control of the size and shape of the cavity and the resultant size and shape of the cement.

In addition to or, in place of, the bone cement for structural support, other ingredients may be included in the material, such as BMP, bone morphogenic proteins, DBM, demineralized bone matrix, BOTOX, and other viral vectors, any bone marrow aspirate, platelet rich plasma, compositie ceramic hydroxyapatite, tricalcium phosphate, glass resin mixtures, resorbable highly purified polylactides/polylactides-co-glycolides and others. U.S. Pat. No. 6,582,439 issued to Sproul on Jun. 24, 2003, incorporated herein by reference, teaches this procedure.

The Reiley et al patent, U.S. Pat. No. 6,248,110, teaches the use of an inflatable balloon within the marrow of most bones in the body, including the vertebrae. The balloon fashions a cavity within the bone as well as providing enough force to adjust the cortical bone to relieve compression or deformation. The cavity and the new contour of the bone may be filled with bone cement There is a possibility of rupture of the balloon within the vertebrae and the escape of the inflating material into the body.

U.S. Pat. No. 6,632,235 to Weikel et al issued on Oct. 14, 2003 teaches the use of an inflatable balloon to be inserted within the vertebral body and expand the space for treatment. The balloon may be removed before the treatment material is injected into the space or the balloon may be a container for the material. There is a possibility of rupture of the balloon within the vertebrae and the escape of the inflating material into the body. U.S. Pat. No. 6,586,859 issued Jul. 1, 2003 to Kornbluh et al teaches the use of electroactive polymers (EAP) as transducers for animating figurines. The polymers act as artificial muscles. The polymers are connected to movable elements of the figure and, upon electrical stimulation, the polymers change shape thereby moving the attached figurine parts.

U.S. Pat. No. 3,731,681 and U.S. Pat. No. 5,176,641 disclose pumps implantable in the body for administering medicaments over long term. The pumps are powered by air pressure or elasticity of a foam to express the medicament from the reservoir. The reservoirs are refillable from outside the body.

An article in the October, 2003 edition of, Scientific American, entitled, “Artificial Muscles,” by Steven Ashley, gives an overview of the research accomplished with electroactive polymers (EAP). The general thrust of the research is the replacement of mechanical, hydraulic and electrical, “actuators,” with polymers that can change shape upon electrical stimulation. The article also suggests that the EAP can expand and contract as well as generate force equivalent to muscle.

Published U.S. Patent application, U.S. 2003/0006669, published Jan. 9, 2003, discloses rolled electroactive polymer (EAP) capacitors, along with the necessary electronic apparatus, bi-directionally used as actuators, sensors and other devices generating mechanical force and strain when electrically excited or generating electrical pulse when mechanically flexed.

The fundamental principals of Maxwell stress and the electroactive polymer (EAP) capacitors are well understood. Basically, the devices are made up of polymeric film, such as dielectric elastomers, with electrodes on both sides forming a capacitor. Electrical energy flowing through the electrodes causes the polymers to deflect along the field lines in compression, when the electrical charges on the opposing electrodes attract each other, and expand perpendicular thereto. Such conversion of electrical energy to mechanical movement is in the nature of a transducer. Of course, the electrodes must be flexible to maintain good contact with the interposed film.

The capacitors also operate in the opposite fashion in that if they are flexed or strained by a mechanical force, the electrodes have different potential producing electrical energy. As a capacitor stores the electrical energy applied to deform it, it releases that charge as it returns to its original shape and size. The change in the size and shape may be used to produce mechanical work and the electrical release may also perform electrical work.

The prior art vertebroplasty systems, such as shown in FIG. 2, include a series of stylets or guide needles to make a pathway from the skin to the cortical wall W of the vertebrae. A cannula 10 is introduced along the pathway and through the cortical wall and a balloon 11 is introduced into the cancellous bone C or marrow. The balloon is introduced into the cancellous bone C in a reduced state and then inflated thereby performing work to create a cavity 12 within the cortical bone by compressing the cancellous bone. The cavity is filled through a cannula with a flowable material, for example, polymethylmethacrylate (PMMA), which becomes rigid. In the case of a collapsed vertebrae, the pressure used in the procedure may be high enough to expand the vertebrae to its original state. Usually, the balloon is inflated with a liquid then deflated and removed before the introduction of the bone cement. However, the balloon may remain as a container for the cement.

Transducers of the prior art, as disclosed by Kornbluh et al, in the form of capacitors, are shown in FIGS. 1A and 1B. The transducer 100 is made up of electrodes 104 and 106 separated by an electroactive polymer film 102. When the transducer of FIG. 1A is electrically charged, it deforms as shown in FIG. 1B. The area increases and the thickness 112 decreases.

The polymer film may be any polymer or rubber or combination thereof that deforms in response to an electrostatic force or whose deformation results in a change in electric field, eg., NuSil CF19-2186 made by NuSil Technology of Carpenteria, Calif., silicone polymers made by Dow Corning of Midland, Mich., acrylic elastomers, VHB 4910 made by 3M Corp. of St. Paul, Minn., polyurethanes, thermoplastic elastomers, pressure-sensitive adhesives, fluoroelastomers, and the like. Thickness may range from 1 micrometer upwards. To increase the deformation capability, the polymer film can be pre-stretched, either directionally or isotropically. Films may be pre-stretched from 100 to 600%.

Differential stretching is also used for special effects. Further, the polymers may be restrained on one or more margins to gain increased deflection in the unrestrained margins. The transducers and polymers are not limited to any particular shape, geometry, or type of deflection. The transducers may be rolled, layered, or folded.

The monolithic transducer has more than one active area on a single EAP. Each active area has a set of electrodes separated by the active area of the polymer. These areas may be arranged to produce a particular result in shape, size, strain or deflection. The electrodes may be of different sizes and the electric charge to different electrodes may differ through charge control circuitry.

Other examples of EAP include electrostrictive polymers, electronic EAP and ionic EAP. Electrostrictive polymers are characterized by the non-linear reaction of an EAP relating to deflection. Electronic EAP change shape or dimensions due to migration of electrons in response electric field, usually dry. Ionic EAP change shape or dimensions due to migration of ions in response to an electric field, usually wet and including an electrolyte. The ionic EAP are usually encapsulated to maintain the environment.

The electrodes are compliant, flexible and expandable to maintain contact with the film during deformation. Suitable materials include graphite, carbon black, colloidal suspensions, thin metals including silver and gold, silver filled and carbon filled gels and polymers, and ionically or electrically conductive polymers. Structured electrodes may also be used, such as, metal traces and charge distribution layers, textured electrodes comprising out of plane dimensions. Also conductive greases, such as carbon or silver greases and other high aspect ratio conductive materials such as carbon fibrils and carbon nanotubes and mixtures of ionically conductive materials.

The electrodes may be subjected to electrical charge through direct wiring coupled with suitable electronics for control of the stress and strain produced by the transducer. The source of the electrical power may be an electrical grid or battery or any other device developing an electrical charge. The electrodes may be charged wirelessly by RF, microwave, ultrasonically or other system. For example, the electric fields may range from 0 v/m to 440 Mv/m and the work output deformation pressure may be 0 Pa to 10 MPa. The transducers are capable of pressures similar to muscle hence the nickname, “Artificial Muscles.”

The transducers include electronic drivers that function to regulate the electrical power supplied to and/or from the electrodes. With regard to the monolithic transducers, the particular active area that is charged and in which sequence may also be controlled. The electronic control system may operate proportionally in that the deflection can be controlled by the electrical power supplied to the capacitor. For example, each transducer may be driven by alternating current or direct current, such as, a dc-dc converter as supplied by EMCO High Voltage of Sutter Creek, Calif., model Q50, with a maximum output of 5 kV and 500 mW of power coupled with a processor such as the PIC18C family of processors made by Microtechnology Inc. of Chandler, Ariz. In order to produce greater pressures the thickness of the EAP may be increased. Other parameters may also be changed individually or collectively, such as changing the dielectric constant of the EAP, decreasing the modulus of elasticity of the EAP, layering multiple EAPs, and others.

### SUMMARY OF THE PRESENT INVENTION

Therefore, an objective of this invention is to provide an electroactive polymer (EAP) in a tool to be used as a surgical instrument to produce work in the body, either singularly or repetitively.

Another objective of this invention is to provide a surgical instrument to produce a cavity within a bone with the instrument remaining in place as a prosthesis or removed to provide space for the introduction of treatment materials.

A further objective of this invention is to provide a cannula with a transducer attached to the leading end.

Yet another objective of this invention is to provide a power source for a surgical instrument for aspiration or infusion of body fluids or medicaments.

### SHORT DESCRIPTION OF THE DRAWINGS

FIG. 1A is a perspective of an electroactive polymer capacitor of the prior art without electrical potential applied;

FIG. 1B is a perspective fo the capacitor of FIG. 1A with electrical bias;

FIG. 2 is a top view, partially in section, of a vertebrae and balloon of the prior art;

FIG. 3 is a perspective, partially in section, of a vertebrae with a cannula and bone tamp of this invention;

FIG. 4 is a top view of a vertebrae an another embodiment of the bone tamp of this invention;

FIG. 5 is a perspective of another embodiment of the bone tamp of this invention;

FIG. 6 is a cross section of an implanted infusion pump of this invention; and

FIG. 7 is a cross section of an implanted aspiration pump of this invention.

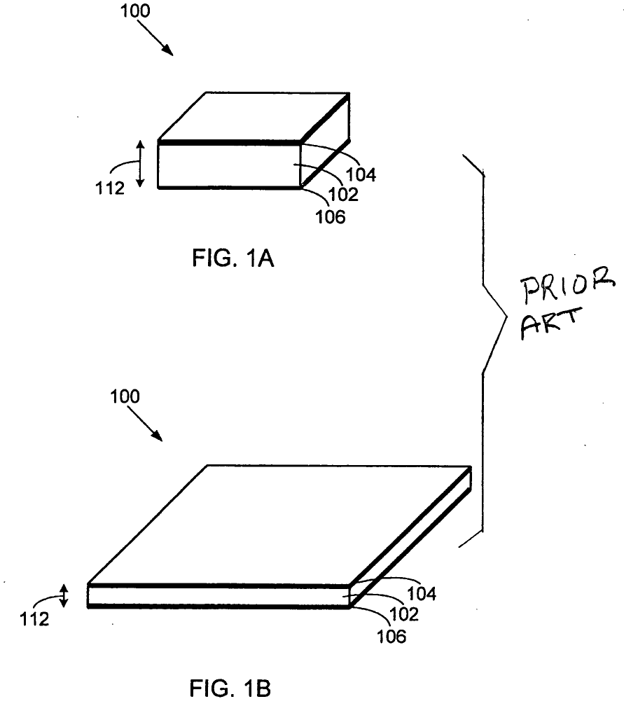

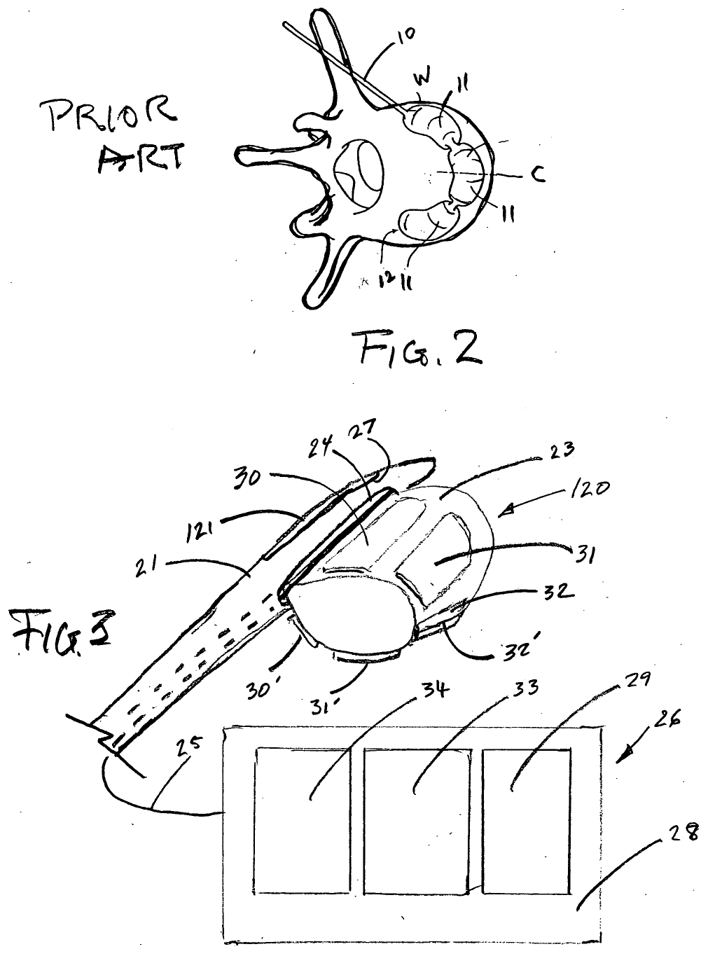

### DETAILED DESCRIPTION OF THE INVENTION

FIG. 3 illustrates one embodiment of a vertebroplasty cannula 21 with a EAP transducer 120 deployed under electrical charge. The transducer 120 is permanently mounted in the cannula and the EPA 23 spans an aperture 24 in the cannula 21. In the initial position, without electrical charge, the transducer is housed within the cannula. The procedure may or may not include a guide cannula (not shown) through which the cannula 21 accesses the cancellous bone area within any skeletal bone. Once the cannula 21 is in a desired location, an electrical charge is directed along cable 25 which connects the transducer, through the cannula, from the electronic control 26 unit. The EPA of the tranducer 120 is deformed by the charge to a second position, as shown in the FIG. 3. The EAP 23 may or may not be pre-strained before attachment about the aperture 24 to increase the deformation. The deformation results in the cancellous bone being compressed or tamped and forming a cavity within the cortical bone. The electrical stimulation is turned off by the control unit 26 and the transducer returns to its first position within the cannula 21. The cannula 21 can then be withdrawn. Another cannula may be inserted through the guide cannula and PMMA or other biological material may be introduced to the cavity.

Because the transducer is initially housed within the cannula 21, the cannula may be introduced without a guiding cannula. Further, the cannula 21 is shown with a second aperture 27 which can house another transducer 121. This transducer 121 may be deployed simultaneously or independently with the first transducer 120 from the control unit. The cannula 21, useful for vertebroplasty or other procedures, may have only one aperture or more than two. The cannula may have multiple bores for introducing or aspirating materials during the procedure, including PMMA, and/or carrying electrical cables.

The transducer 120, as shown, is a monolithic transducer in that it has only one EAP 23 between separate electrodes 30, 30′; 31, 31′ and 32, 32′ forming several active areas. These electrodes may be excited in various sequences or simultaneously by control unit 26. The electrodes may produce differing effects because of each shape or the electrical charge.

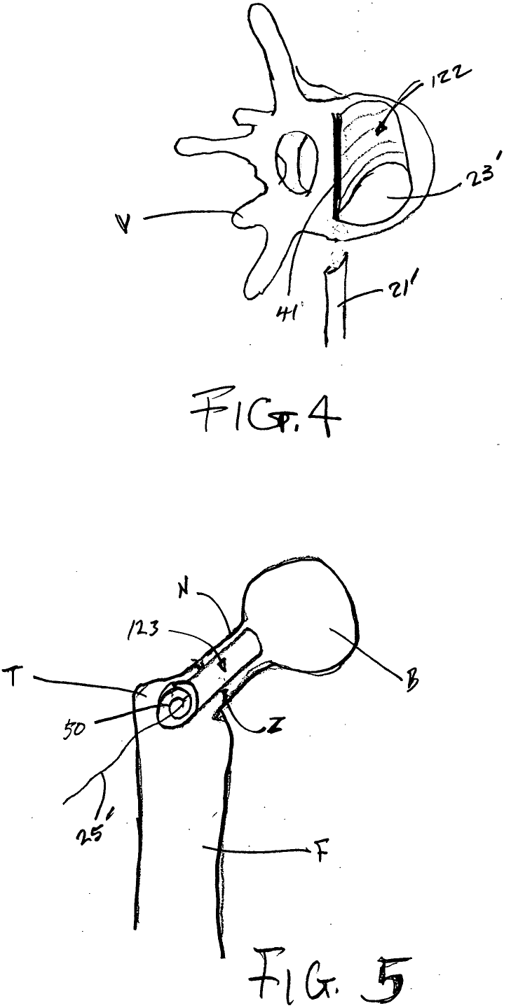

The control unit 26 includes a processor 28 or computer for over all command and control. Depending on the electrical power source, there may be converters, transformers or other modifying components. The control unit includes conditioning electronics 29 to provide or receive electrical energy from the electrodes and function as stiffness control, energy dissipation, electrical energy generation, polymer actuation, polymer deflection sensing, and control logic. The electrical source may be a battery with 1 to 15 volts with step up circuitry 33. There is step down circuitry 34 to adjust the voltage from the transducer(s). The system may be operated with alternating current. Another bone tamp is shown in FIG. 4 disposed within the cortical bone of a vertebrae V. The transducer 122 has an electrode on each side of the EAP 23′. One margin of the EAP is fixed on a frame 41 to prevent deflection. The transducer may be arranged to deflect into different shapes and sizes either by fabrication or by electrical stimulation. As shown, the transducer 23′ is in the second position approximating a wedge. The other margins are shown as straight but could be curved or angled or a combination of both. A cannula 21′ is shown as withdrawn from the cancellous bone. The cannula 21′ may be introduced into the cancellous bone over a docking needle. The transducer is then inserted after the needle is removed from the cannula. The cannula delivers the transducer in the initial position with the EAP 23′ folded or wrapped about the frame 41. The frame serves as a limiting margin of the cavity to be formed in the vertebrae. Under the influence of electrical energy, the transducer deforms to the second position, shown. The transducer 122 may be controlled, monitored and charged wirelessly from outside the body or by cable. After the cavity has been formed, the power is stopped and the transducer returns to the first position. In vertebroplasty, the expansion of the transducer is such that the end plates of the crushed vertebrae are displaced to a more normal location. Bone cement and/or other materials may be injected into the cavity with the transducer in place. Of course, the transducer may be removed by cannula before the introduction of the materials, if desired.

In FIG. 5, another bone tamp is shown inserted into the neck N of the femur F. The neck is that portion of the femur that extends between the trochanter T and the ball B. A fracture Z of the neck of the femur is common in older people and is difficult to immobilize. A transducer 123 in the form of an internal splint is introduced into the cancellous bone of the neck N in the initial position by cannula. The transducer 123 is pre-stretched about a spring 50 to maintain the stretch and to direct the deformation. The transducer 123 may be charged by cable 25′or by RF (radio frequency energy). The transducer assumes the second position and expands against the cortical wall forming an internal splint.

The transducers may be used for other purposes within the body. For example, FIG. 6 illustrates an implantable infusion pump 60 inserted beneath the skin S. The transducer 124 is contained within a sheath 61 which serves to separate the transducer from the medicament to be delivered by the pump. The transducer may be wound around a spring or frame that allows expansion and contraction in the longitudinal axis. The sheath may be elastic to expand with the transducer when the electrical charge is applied through the cable 62. The sheath may be inelastic but sized to accommodate the expanded transducer. The transducer is enclosed within an inelastic sheath. Either sheath may contain a liquid with an electrolyte and the transducer may be ionic. As shown, the transducer 124 and sheath 61 are in the expanded second position with the medicament expressed through the exhaust port 64 from the reservoir 63.

The external wall of the pump has a self sealing refill port 67 penetratable by hypodermic needle 69 to resupply the reservoir when the transducer is in the initial position. The transducer 124 is of the type that resumes the initial position upon cessation of electrical power. A one-way valve 65 controls the flow of the medicament from the reservoir to the body from the port 64 through the catheter 68. The one-way valve may be a slide valve, a flapper valve, a ball valve or other device. The pump casing 66 is a bio-acceptable material, usually a polymer with a smooth external wall. The pump may be used in a timed sequence with the transducer slowly expanding over time and then returning to the initial position for the reservoir to be refilled.

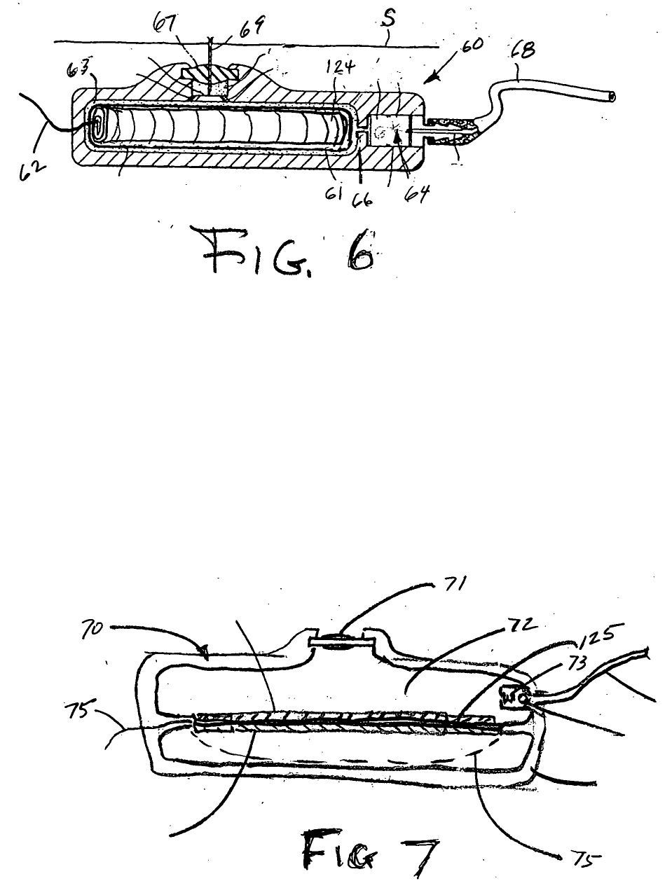

Another pump is illustrated in FIG. 7. As shown, the pump is an aspirator for withdrawing materials from the body. The aspirator pump 70 has a smooth body for implantation within the body with a self sealing port 71 for withdrawing collected materials from the pump reservoir 72. The pump has a one-way valve 73 for controlling flow into the pump from a catheter 74. The transducer 125 extends across the reservoir 72 as a diaphragm and bottom wall. As the electrical charge is applied through cable 75, the transducer will deform into the lower chamber 75 of the pump body producing a negative pressure in the reservoir 72. The negative pressure may be monitored and controlled over time by the electronic control system. Upon cessation of the electrical stimulation, the transducer will return to the original position.

Of course, both pumps will operate outside the body and when the one-way valves are reversed perform the opposite function as that described above.

A number of embodiments of the present invention have been described. Nevertheless, it will be understood that various modifications may be made without departing from the spirit and scope of the invention. Accordingly, it is to be understood that the invention is not to be limited by the specific illustrated embodiment but only by the scope of the appended claims.

1. A surgical device for producing work in the body comprising an implantable unit including a transducer, said transducer having a dielectric polymer film disposed between two electrodes, said electrodes connected to an electrical lead, said film having an initial position with a first size and an excited position with a different second size, said transducer accomplishing work resulting from said film transitioning from said first position to said second position.

2. A surgical device of claim 1 wherein said transducer is adapted to be placed within a bone with said film in said initial position, said film expanding to said second different position by electrical impulse applied to said electrodes through said lead, said film returning to said first position upon cessation of said electrical impulse whereby said transducer forms an internal cavity in the bone.

3. A surgical device of claim 1 wherein said transducer is adapted to be placed within a bone with said film in said initial position, said film expanding to said second different position by electrical impulse applied to said electrodes through said lead whereby said transducer forms an internal splint in the bone.

4. A surgical device of claim 1 wherein a pump is adapted to be implanted in the body, said pump having an output port connected to a flexible reservoir of variable volume, said reservoir adapted to be filled with a medicament, said reservoir having a refill port for percutaneous refilling of said reservoir, said transducer in contact with said reservoir, said film reducing the volume of said reservoir when transitioning from said first position to said second position.

5. A surgical device of claim 4 wherein said reservoir is adapted to be empty, said outlet port is adapted to receive body fluids, said transducer in contact with said reservoir with said film in said second position, said film transitioning from said second position to said first position as said reservoir fills, said change in position generating an electrical impulse through said electrodes and said electrical lead.

6. A surgical device of claim 1 wherein said transducer is adapted to be inserted in the intervertebral disk space in said second position, said capacitor accomplishing work by transitioning from said first position to said second position by compression of said intervertebral space and generating electrical impulse through said lead.

7. In an orthopedic system having a guide wire for percutaneous penetration and insertion through a bone having a cortical portion and a cancellous portion, a cannula for telescoping insertion along said guide wire and penetration into the cancellous portion of the bone, the improvement comprising an electroactive polymer sandwiched between electrodes with an initial position of a size and shape for insertion through said cannula into the cancellous portion of the bone, said electroactive polymer having malleable properties including changes of size and shape, a source of electrical energy for stimulating said electrodes, said electrodes having an electrical connection with said source whereby activation of said source excites said electrodes causing said malleable properties to change at least one of said size and said shape of said electroactive polymer and alter the bone.

8. In an orthopedic system of claim 7 wherein at least one of said size and said shape of said electroactive polymer expands and alters the cancellous portion to produce a cavity in said cancellous portion.

9. In an orthopedic system of claim 7 wherein upon cessation of said electrical stimulation said electrodes returns said electroactive polymer to said initial position.

10. In an orthopedic system of claim 7 wherein at least one of said size and said shape expands to alter said cortical portion of the bone.

11. In an orthopedic system of claim 8 wherein upon cessation of said electrical stimulation said electroactive polymer returns to said initial position.

12. A transducer for insertion into a bone for altering the cancellous portion comprising an electroactive polymer with malleable properties sandwiched between opposing electrodes, said electroactive polymer having an initial position of a size and shape to be inserted into the cancellous portion of a bone, said malleable properties of said electroactive polymer changing in response to electrical stimulation, and an electrical energy source, said electrodes electrically connected to said source whereby activation of said source results in changed properties of said electroactive polymer and altered bone.

13. In an orthopedic system of claim 12 wherein upon cessation of said electrical stimulation said electroactive polymer returns to said initial position.

14. A method of forming a cavity within a bone having a cortical body and a cancellous interior comprising the steps of
a) inserting a cannula through the cortical body of a bone into said cancellous interior, said cannula including an aperture, a transducer spanning said aperture, said transducer having an initial position and a second position,
b) connecting said transducer to a source of electrical energy, and
c) said transducer transitioning to said second position, said second position being larger than said initial position whereby said cancellous interior is compressed to form a cavity.

15. A method of forming a cavity of claim 14 comprising the steps of
a) providing a separate transducer with a frame,
b) depositing said transducer in said cancellous interior in said initial position, and
c) withdrawing said canulla.

16. A method of infusing a medicament from a variable volume pump comprising the steps of
a) providing a pump having a body with a reservoir and an infusion port connecting said reservoir with said exterior of said body, a flexible diaphragm connected to said body in said reservoir separating said reservoir into two chambers, a transducer in one chamber, and a medicament is said second chamber,
b) said transducer having an initial position and a second position,
c) applying an electrical charge to said transducer,
d) said transducer transitioning from said initial position to said second larger position whereby said first chamber is enlarged and said second chamber is decreased and said medicament is expressed from said infusion port.

17. A method of infusing a medicament of claim 16 comprising the steps of
a) providing said transducer as said diaphragm.
